(19) United States
(12) Patent Application Publication
(10) Pub. No.: US 2012/0053494 A1
(43) Pub. Date: Mar. 1, 2012

(54) Method And Apparatus For Altering Activity Of Tissue Layers

(76) Inventor: Yoni Iger, Haifa (IL)

(21) Appl. No.: 13/220,892

(22) Filed: Aug. 30, 2011

Related U.S. Application Data
(63) Continuation of application No. 10/505,017, filed on Aug. 18, 2004.

(30) Foreign Application Priority Data
Mar. 30, 2002 (IL) 148791

(51) Int. Cl. A61N 7/00

(52) U.S. Cl. 601/2

(57) ABSTRACT

The present invention concerns ultrasonic methods and devices for altering activity of layers of natural- or of artificial tissues and organs, and for altering activity of particular components within said layers, while minimizing alterations in neighboring layers located deeper to—or outer to—treated layer.

Focused or non focused irradiation at certain angles and preferably via cooling medium, at least partially creates surface waves propagating in the appropriate layers, and alters their activity, while leaving the other layers essentially intact. Monitoring of beam location may be provided.

The present invention may be constructed for either superficial treatment, or minimal invasive treatment, of layered tissues and organs and may be either stand alone or added to another device in cosmetic and clinical applications

### CROSS-REFERENCE TO RELATED ACTIONS

This application claims priority to and is a continuation of U.S. patent application Ser. No. 10/505,017, filed Aug. 18, 2004, which is incorporated by reference herein in its entirety.

### FIELD OF THE INVENTION

The present invention generally concerns methods and devices for altering biological activity using mechanical vibrations, and more particularly to ultrasonic methods and devices for altering activity of tissue- or of organ layers, and for altering activity of particular components within said tissue or organ layers, while minimizing alterations in neighboring tissue or organ layers located deeper to—or outer to—treated layer.

### BACKGROUND OF THE INVENTION

The human body, as the body of other multi-cellular living creatures, is made of numerous cell types, forming tissues and organs, or organ components. Each of the tissues and each of the organs is mostly arranged as multi layer tubular or flat structure. For instance blood vessels, digestive tract, fragments of the respiratory system, long bones or uterus are multi layered tubular structures, whereas the skin is an example of a multi layer flat structure.

The term “layer” according to this invention refers to a morphologically or histologically distinguished layer, which compose a layered part of a tissue or of an organ. A layer might be composed also of several morphological layers having different cell types, for instance the dermal zone of the skin, or being composed of bulk of a singled cell type, for instance the smooth muscle cells of the coronary arteries. Essentially, there are differences in the density, elasticity and other mechanical properties of the different layers of a tissue-, or of an organ structure. For instance, there is a clear mechanical difference between the blood liquid located at the blood-vessel lumen, and between the more rigid vessel wall. In the uterus, the endometrium which is a rather loose tissue loaded with angiogenetic processes and blood, differs in its mechanical properties from the deeper compact musculature bulk of the uterus. In the skin the dermal zone, compose largely of collagen fibers,

significantly differ in its mechanical properties from both superficial epidermis and from the deeper subcutaneous adipose zone, composed largely of fat cells.

At times it is desired to affect part of, or an entire layer of tissue or organ at particular body zone, or to affect particular components of certain layer without affecting the surrounding tissue- or organ layers. This is a common need in both medical and cosmetic arenas.

In the cardiovascular system, for instance, the stenotic and the restenotic narrowing and the subsequent obliteration or mal performance of the coronary arteries cause significant morbidity and mortality. It requires bypass surgery or minimal invasive manipulations with angioplasty and stents for stenosis, and much less treatment options during the restenosis phase. It is then, or preferably even earlier as preventative procedure, desired to selectively and precisely affect the entire tubular layer of the smooth muscle cells of the tubular coronary blood vessels, so to prevent or to cease the restenosis, without affecting the lumenal endothelial cells,

In the digestive tract it is occasionally essential to selectively affect benign polyps, so to prevent cancerous transformations, without affecting the towards-lumen cover of moist epithelial tissue. In the uterus it is occasionally needed to affect the endometrium facing the uterus lumen, so to cease excessive menstrual bleeding in a non-ablative manner and without affecting the deeper uterus tissues.

In the skin it might be needed to affect certain components located in particular layers, for instance hair follicles, pigment cells, or blood capillaries. It might be further needed to affect entire layers of the skin, for instance affecting the epidermal layer for juvenile look, or affecting the dermal zone for wrinkle removal.

In the respiratory system, it might be needed to affect obstacles of the pulmonary bronchi, without harmfully affecting the alveoli or pneumocytes, and without initiating hemorrhage due to harmful effect of cavitation an the air spaces.

Several methods and devices have been developed for affecting tissues, organs and their components. Said methods largely emphasize on non- to minimal invasive procedures using different energy sources, including laser, microwave, radio frequency, or ultrasound as affective elements. These methods, however, do not create selective effect derived from the different mechanical properties of the different layers, to create the create layer restricted desired effect.

When therapeutic ultrasound is used, the tissues in the beam path absorb energy and suffer damage, which otherwise can be employed to affect the particular desired location of effect. According to the teaching of U.S. Pat. No. 6,206,843 one way to solve this excessive damage, for instance when treating blood vessels, is by applying acoustic pressure and subsequently ablative procedure at reduced time and intensity and with less side effects.

At times, it is desired to cause alterations in certain layer of multi layered tissues or organs, without affecting layers located deeper or superficial to treated zone.

### SUMMARY OF THE INVENTION

The present invention concerns a method and device for ultrasonically affecting and causing alteration of a certain layer of a multi layered tissue or organ, in a non invasive or minimally invasive procedures, and with minor effects an other layers of the said treated tissue or organ. It is performed by using the different mechanical properties of the different tissue or organ layers, and the subsequent difference in the relevant sound velocity of the different layers, as selective element for guiding the ultrasonic waves. The mechanical differences between layers, and between layers and the ambient media, combined with leading element of irradiation angle, enable shifting of the waves towards surface waves running in particular layers at least partially parallel or almost parallel to the surface, and therefore affecting particular layers parallel to the tissue or organ surface without affecting, e.g., deeper tissues. During irradiation surface is preferably being cooled, enabling creating effects, including thermal effects, at layers deeper to the surface leaving the surface intact.

When ultrasonic irradiation hits structure at an oblique angle, beam is split to three major components, reflected longitudinal waves, transmitted longitudinal waves and shear waves. Whereas the reflected angle of the waves is always equal to the irradiation incident angle, the angle of the refracted waves is determined by the properties of the two media, in particular by the sound speed in bath media. The direction of propagation of the transmitted waves in the structure therefore changes and is different from the direction or angle of irradiation.

In general, when irradiating an object at a certain angle between a perpendicular axis to the object and the irradiation farce, and from a zone A having certain characteristic of sound velocity, into another zone B (the object) characterized by a higher sound velocity, the refracted irradiated ultrasonic waves in the object will tend to run in an angle higher than the irradiation angle, towards running at least partially in parallel to the object surface.

For instance when irradiating from water (sound velocity of about 1500 m/sec) into the skin (sound velocity of about 1,700 m/sec), at an angle greater than about 60 degrees with respect to perpendicular axis, then majority of the refracted waves will run parallel, or almost parallel to the skin surface, as surface waves. Typically this said angle of 60 degrees is a critical angle for the skin, where most longitudinal waves transferred into interface nature, a rather mixed mode of longitudinal and of shear waves. This combined mode is also characterized by high attenuation and therefore create demarcated effect at attenuation zone, and essentially do not penetrate or affect other locations. Said interface waves, at least partially run in parallel to the surface, and therefore termed surface waves. When treating layered tissues or organs it provides significant advantage to treat particular layers without affecting the ambient layers, outer to or deeper to treatment zone.

A common way to create surface waves, in particular in the form of shear waves, is to transmit longitudinal waves through water or gel. Water does not support shear waves and therefore at the interface, between the water and the irradiated object, there is a reflected longitudinal back into the water and two wave modes refracted into the irradiated object—a longitudinal and is a shear wave. So the water liquid serves as essential element in the creation of these shear waves. The other way to create shear waves is by using a probe which directly excites shear waves. In order to transmit these shear waves into the irradiated object, the coupling between the probe and the said object must be a liquid of a very high viscosity, or a sticky solid.

It shall be noted, however, that waves running in parallel to the surface can be also of other mode, including purely longitudinal waves. This can be done, for instance by irradiating the object in a certain direction essentially parallel to the object surface. This irradiation is carried out while considering the natural morphology of the object, or alternatively with artificially modified morphology of the irradiated object.

When ultrasonic irradiation to a structure is originated from more than one ultrasonic source, the total disturbance at each point of the irradiated structure is the vectorial sum of the different particular mechanical disturbances produced by each ultrasonic wave source.

The static forces which are created when ultrasonic waves undergo changes in their direction (or in amplitude) further induce radiation pressure. Surface waves, however, as well as the combined interface mode, can produce all effects that can be created by regular longitudinal waves, including thermal and non thermal effects, heating and ablation, cavitation, microstreaming and shear stress, pressure, radiation force, torque and streaming.

According to a first aspect, the present invention concerns a method for affecting superficial layers of tubular or flat tissues or organs comprising: applying to said tissues or organs ultrasonic irradiation at an angle which produces ultrasonic waves at least a portion of which propagates in said superficial layers and at least partially in parallel to the skin surface. The procedure can be carried out as stand alone, or be combined by another ultrasonic irradiation, perpendicular or in another angle to the said tissue or organ, so to have a constructive vectorial sum of effects. The oblique irradiation, source of the affecting surface waves, might be combined also with another source of energy irradiation.

The term “ultrasonic surface waves” in the context of the present invention, refers to ultrasound waves which direction of propagation is at least partially, and preferably largely, parallel to that of the tissue- or of the organ surface, as the case might be. The surface waves may be produced by a combination of different wave modes, however at least one of the following wave modes should constitute the surface waves: longitudinal waves, shear waves, bending waves or torsion waves.

By one embodiment of this aspect, the propagation of the ultrasonic surface waves is precisely at the organ surface, e.g., at the interface between the uterus endometrium and the inner uterus lumen. However, in accordance with a preferred embodiment of the invention, the propagation of the ultrasonic surface waves is carried inside the endometrium, a short distance deeper than its lumenal surface. Such an embodiment ensures that the main effects of the ultrasonic energy, are achieved in a certain layer of a specific depth of the uterus where appropriate destructive effect is needed, while deeper layers are not penetrated by the energy and therefore do not absorb it and are not affected. The depth of the treated layer is typically between several mm to tens mm.

By another embodiment of this aspect, the propagation of the surface wave is deeper to the organ surface, e.g., at the smooth muscle layer of a coronary artery. In accordance with this embodiment of the invention, the irradiation is from the vessel lumen and in direction of the length axis of the vessel. The propagation of the ultrasonic waves in the target is carried out at the muscle layer of the vessel, leaving the internal endothelial cells intact. In this embodiment, the blood inside the vessel is used as both ultrasonic coupling agent, and as cooling liquid, further provides heat protection for the inner endothelial cells by conduction and convection. Irradiation is in a minimally invasive procedure.

By another embodiment of this aspect, the propagation of the surface waves is through the entire layers of the organ, e.g., at the wall of the urine bladder. In accordance with this embodiment of the invention, the irradiation is along the three major layers of the bladder to create there an effect, leaving the surrounding tissue and the content of the bladder intact. Irradiation is in a minimally invasive procedure.

By another embodiment of this aspect, the method is used as add-on to another method to achieve synergic effect. According to the teaching of pending application PCT IL99/00533, the ultrasound waves are transmitted at a rather high intensity, along hair shafts by using the hair as wave guides, till dissipation at the follicle, for hair removal. According to the present invention, ultrasonic waves are transmitted as surface waves and reach the hair follicle at an angle which is essentially perpendicular to the direction of the growth of the hair shaft. A combined method suggests irradiation of surface waves together with irradiation via the hair shaft at lower intensity. Ultrasonic characteristics for the two irradiations shall be those that when combined together the total disturbance at the follicle area of the hair will be constructive. Furthermore, the combined method will advantage the different sensitivity of skin's components to heat. The follicle cells (which are of epidermal origin) are sensitive to heat and will receive vectorial combination of the two irradiations. Concomitantly, the collagen fibers which covers the follicles invaginations, are much less sensitive and will anyhow receive only the surface wave irradiation. This will provide improved hair removal.

It shall be noted that the perpendicular direction of the waves ensures that energy irradiation has only a superficial effect, since the wave propagation which is perpendicular to follicles is also parallel to skin surface, and therefore essentially energy does not propagate to deeper regions of the skin and also the skin surface remains intact. The second irradiation source, in addition to the surface waves, might be virtually any other type including laser, RF, microwave and the like.

In order to minimize side effects to the irradiated tissue or organ, a cooling agent might be applied to the said tissue or organ, so that the surface of the treated zone is cooled while the effects, including heat, are built in the appropriate deeper layers of the treated zone. Examples of cooling agents are: water or cooling gels. According to the treatment site, cooling agents might be also body natural fluids such as blood, urine and the like. Under both circumstances the cooling agents in addition to convection and conduction of heat, might serve also in the actual creation of the surface waves, as explained above. Altering parameters, such as cooling temperature, might affect depth and intensity of the created biological alteration.

The angle of irradiation used to produce mainly surface waves should be calculated and chosen empirically, while considering mainly the characteristics of the media the waves travel in, and also the mechanical characteristics of the system, the cooling medium and ultrasound irradiation parameters. The frequency or intensity of the ultrasound do not affect the critical angle for the creation of surface waves. Surface waves might be created also by modifying the morphology of irradiated tissue or organ so waves will run initially in parallel to the surface.

The ultrasonic parameters used with the appropriate surface waves are interconnected, so that when reducing intensity at certain frequency, the reduction of total energy can be compensated by increasing duration of the energy application; or when increasing frequency, resulting in increase in flow of energy (assuming that both reach desired depth), the increase may be compensated by decrease in intensity treatment duration etc. Focused beam, either regular or by phase array might be used to increase irradiation intensity of a layer, whereas increased frequency might be used to reduce the travel length of the waves in a layer.

The parameters required for creation alterations in layers of tissues or organs in accordance with the surface wave aspect of the invention are as follows:

Frequency: 20 kHz-50 MHz, preferably 200 kHz-3 MHz;

Intensity: 0.001 Watt-1000 Watts, preferably 0.1-5 Watt or several hundreds Watt for regular and focused beam, respectively;

Duration: 0.001 sec.-10 min., preferably split of second till seconds, and tens of seconds till few minutes for focused beam and for regular beam, respectively.

Mode: pulsative or continuous.

### BRIEF DESCRIPTION OF THE DRAWINGS

For a better understanding of the invention and further methods devices and features thereof, reference is made to the attached non limiting drawings wherein:

FIG. 1 shows angles of ultrasound irradiation required for production of surface waves in accordance with the invention;

FIG. 2 shows a schematic device and system for carrying out the method of the invention; and

FIG. 3 shows another embodiment of a system for carrying out the method of the invention for minimally invasive procedure.

### DETAILED DESCRIPTION OF PREFERRED EMBODIMENTS

Therapeutic ultrasound partially affects via temperature elevation. The temperature increase of a medium through which ultrasound wave propagate is a result of the absorbance accompanied by the acoustic beam attenuation. Absorbance and attenuation are higher when frequency is higher, and accordingly the intensity of the acoustic beam reduces exponentially along the beam path. Intensity is significantly reduced also when using shear waves or interface waves. The former are waves of shear stress along the plane of wave propagation which attenuates up to four order faster than longitudinal waves, which are waves that propagate with compression and decompression along the direction of wave propagation. The interface waves are combined mode of essentially longitudinal and stress waves, having combined characteristics.

Ultrasonic irradiation being applied to biological object in a direction oblique to the surface, can produce different wave types. It can produce non surface waves, including longitudinal and shear waves which might penetrate deeper into the tissue. Concomitantly, and occasionally alternatively, it can produce mixed mode of two first types that essentially run in parallel to the skin surface, termed surface waves.

The surface waves become a significant part of the total propagated energy, when angle between perpendicular axis and the irradiation axis is higher than a critical angle. It can be presented also as the angle between irradiation axis and the irradiated biological object, and then angle shall be smaller than a critical angle. Mostly the range of critical angle between biological surface and wave front is in the range of 10-30 degrees (equivalent to 60-80 degrees from perpendicular axis), but it largely depends on the mechanical properties of the media.

There are two extreme cases. At one case and certain angle most of the energy is featured as surface waves. At this angle longitudinal waves essentially do not penetrate the depth of the object, but partly reflected, and mostly creates together with shear waves the surface waves. For instance, for the skin at the angles of 10-30 from the skin, most of the waves will be surface waves, whereas above 30 most waves will penetrate into deeper layers of the skin. The other extreme case is below about 5 degrees between irradiating force and object (e.g., the skin), where most of the energy will be reflected from the skin object.

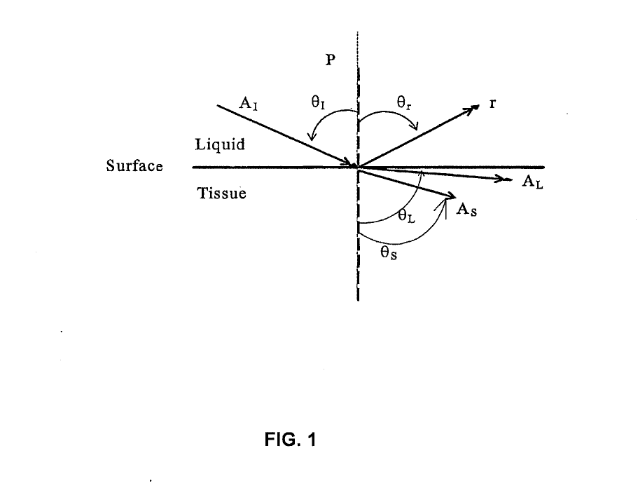

Shear forces are created by propagation of the shear waves. The method consists of the transmission of longitudinal waves Aᵢ through liquid or gel. Liquid, such as water, does not support shear waves, whereas solids does. Therefore at the surface interface, between the water and skin, there is a reflected longitudinal wave r into the water and two refracted waves into the skin. One wave is longitudinal A_{L} and the other one is a shear wave Aₛ, as shown in FIG. 1.

The intensity partition, between the said two refracted waves in the skin depends on the angle between the irradiation source and the perpendicular axis, incident angle θr, and the physical properties of the liquid and the tissue. The physical properties of the liquid, water for example, and of the tissue, skin for example, are known (for instance at: Nyborg L.N. Biological effects of ultrasound: mechanisms and clinical applications, NCRP pub., Bethesda, Md. (1983)). The longitudinal velocity for skin is given as cₗ=1720±45 m/s, and the density ρ=924±24 kg/m³. An average of 60 ratios of cₛ/cₗ of different materials gives cₛ/cₗ=0.51±0.8 and accordingly we estimated cₛ=880 m/s. For water following characteristics were taken: c_{w}=1487 m/s and ρ=999 kg/m³, all at a temperature of 25° C. Relation between incident angle—θᵢ and angles of a reflected longitudinal θᵣ, transmitted longitudinal θ_{L} and transmitted shear θₛ wave were found from the next Snell equation relation:

$$\frac{{c}_{ω}}{sin {θ}_{I}} = \frac{{c}_{ω}}{sin {θ}_{r}} = \frac{{c}_{ω}}{sin {θ}_{L}} = \frac{{c}_{s}}{sin {θ}_{s}}$$

Angles θ_{I}, θᵣ, θ_{L} and θₛ are all measured in perpendicular to the tissue, the skin surface in this example. θ_{I} is always equal to θᵣ. The incident angle is called “critical angle” θ_{c} when c_{w}/(cₗ*sin(θᵢ)>1, because at angles above this angle, the transmitted longitudinal wave does not penetrate the skin surface but reflected from it. At this angle, however, the longitudinal waves essentially run in parallel to the skin surface. For the above mentioned sound velocities in this non-limiting example, θ_{c} is equal to 69 degrees. Directions of a reflected longitudinal and transmitted longitudinal and shear waves at the critical angle are demonstrated in FIG. 1. At the critical angle an angle between skin surface and transmitted longitudinal wave is 5.5 degrees, i.e., almost parallel to the skin surface, and angle of transmitted shear waves is 54 degrees.

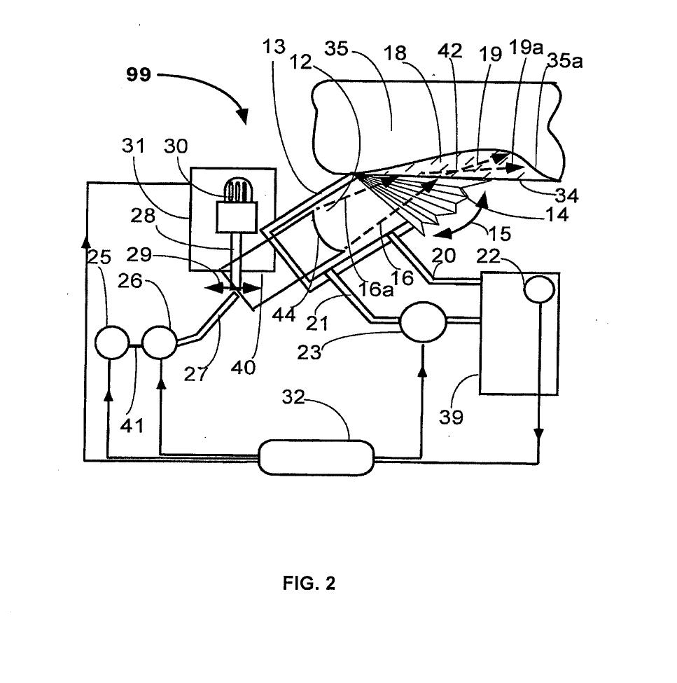

FIG. 2 is a non limiting example of system 99 for altering activity of superficial layer by application of ultrasonically created surface waves to the treated area. The irradiating element, a focusing transducer 40 having concave irradiating surface 44, is located in the container 13, for instance a cylinder, which is filled with degassed water to prevent non desired cavitation in the media outside of treated object 35. The cylinder end is a flexible concertina-like sleeve 14, which can be changed in the general direction of arrow 15. Flexion of the sleeve 14 changes angle between schematic focused beam 12, with borders of wave propagation 16 and 16a, and between the target surface 34. Said angle can be calibrated accordingly to reach the appropriate angle so to initiate desired effect at desired layers.

After impinging surface 34 waves propagate in general direction of arrows 19 and 19a, having focus zone 42 at treated area. Affected layer 18 is located at superficial layer of treated zone 35, and can be distinguished by schematic border of effect 35a.

Degassed water in the container 13 circulates through tubes 20 and 21 to, and from water container 39, by pump 23. Circulated water serve both as a cooling agent as well as an ultrasonic coupling agent. Control unit 22 regulates water temperature and flow rate of the circulated water, by affecting pump 23 and potential cooling unit (not shown). Due to the possible cooling effect, and if and when the layer alteration source is thermal, surface of zone 34 can remains cooled and intact whereas profile of elevated temperature is built deeper in the treated zone. However, cooling is only a preferred embodiment, and water might be served only as coupling agent.

Signal generator 25 and amplifier 26, are connected with each other and with transducer 40 by appropriate cables 41 and 27 respectively. Optionally both signal generator and amplifier can be constructed as an integral component enabling changing frequency and intensity of ultrasonic irradiation.

To reach homogenous skin treatment without differences between ultrasonic areas of maximas/minimas, the container 13 and the transducer 40 can be slightly moved by driver 28 in the direction of arrow 29. Mechanical force-creating element 30, which might be for instance a motor, electromagnet or ultrasonic probe, is mounted into device body 31 and causes the motion of driver 28.

Control of the different controllable processes, such as ultrasonic signal generation, water pump, movement of transducer, angle of irradiation and the like is carried out by central control unit 32, which simultaneously may receive data from different controllers.

The system might contain also on-line monitoring unit to locate location of beam and effect created at desired layer. Monitoring can be performed with the same ultrasonic device, for instance when working alternately between affecting and monitoring phases, and data obtained is further analyzed and possibly stored and implanted by the control unit 32.

The system might further contain means for continuous movement over the desired location. Optionally the entire system, possibly with reduced cooling effect, might be integrated into a hand held consumer device.

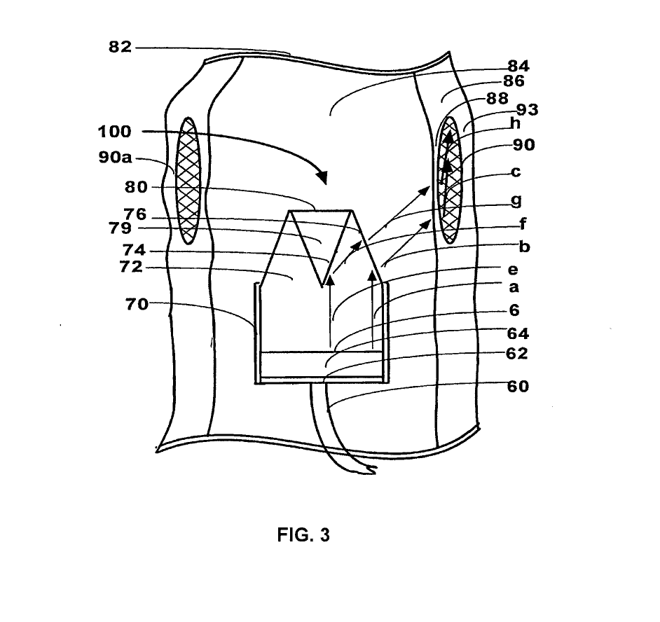

FIG. 3 is a non limiting example of device 100 for radial irradiation, distal part of the entire system, for altering activity of a layer located in middle-depth of a tubular organ, part of which is schematically given in cross section as target 82. Said target is composed of lumen 84 with natural body fluids, and of organ wall 86. The device is preferably inserted to desired location using conventional guiding means, for instance a catheter.

Signal transmitted via cable 60 deliver signal to activate ultrasonic creating mean, probe or transducer 64, via matching 62. Said ultrasonic creating mean irradiates ultrasonic waves that propagate via horn 72, and from there into the tissue. Irradiation can be performed also without the horn, providing that the irradiating source is in appropriate angle towards the target wall 86. However, the horn is partially covered by ultrasonic absorbing medium 70, preventing irradiation from non appropriate parts and directions of the irradiation device 100.

Schematic ultrasonic wave a, irradiated from surface 68 of the ultrasonic creating mean 64, propagates towards oblique device wall 76, being refracted into lumen 84 as wave b, impinges inner side of organ wall 86, being further refracted and propagates as surface, or close to surface wave c in said organ wall 86. Another schematic wave e impinges oblique device-wall 74. Space 79 located between cone margins 74 (radial, as the entire device in this example) and distal wall 80 is composed of material of high ultrasonic attenuation, such as air, and therefore surface 74 acts as reflector. Schematic wave e is reflected from wall 74, and continues as schematic wave f. The later propagates, impinge wall 76, refracted and continues as wave g, which further propagates, impinge and refracted at inner side of organ wall 86 in the general direction of schematic wave h.

The vectorial contribution of schematic waves c and h, in organ wall 86, creates demarcated layered effect, along the long axis of tubular organ 82. Affected zones 90, and 90a, which actually refer to the same affected radial zone, are located in mid layer of organ wall 86. The combined mode of both reflected waves, and waves directly refracted into the same location, increases the local effect. Assuming heat initiated effect, area 88, in the lumenal side of organ wall 86 remains intact due to the convection and conduction of heat by the natural body fluids of the lumen. Area 93 remains intact since waves propagates in the organ wall 86 in particular in parallel to—or close to parallel to the surface, and without essentially propagating into and affecting deeper layers.

While there have been shown preferred embodiments of ultrasonic methods and devices for altering activity of layers of tissues and of organs, it is to be understood that many changes may be made therein without departing from the spirit of the invention. The invention embraces any and all changes, modifications, alternatives or rearrangements of the method and device as defined by the claims, including the use of method and device for non-biological structures.

1. A system for altering activity of at least a portion of one or more layers of biological tissue through application of ultrasound waves, the system comprising:
an ultrasound transducer having an irradiating surface and being constructed and designed to emit ultrasound waves;
a housing container including an interior, the housing container configured to dispose within its interior at least a portion of the ultrasound transducer including the irradiating surface and to permit ultrasound waves to emit from the irradiating surface towards and through a first end of the housing container towards the biological tissue;
a mechanical force-creating element operatively connected to a portion of the ultrasound transducer that extends from a second and opposite end of the housing container, the element being constructed and designed to move the container and the ultrasound transducer during operation of the ultrasound transducer along the biological tissue;
a fluid container joined with the housing container such that the interior of the fluid container is in fluid communication with the interior of the housing container, the fluid container being constructed and designed to hold and to circulate a given volume of a fluid within the interior of the housing container, the fluid providing at least one of ultrasound coupling and cooling; and
a sleeve on the first end of the housing container through which ultrasound waves emit, the sleeve being configured as singularly adjustable relative to a surface of the biological tissue such that ultrasound waves strike the surface of the biological tissue at angles at least within a range between an irradiation axis of emitted ultrasound waves and an axis perpendicular to the surface of the target tissue so that at least a portion of ultrasound waves convert to and impinge the target tissue as surface waves that propagate substantially parallel to the surface of the target tissue.

2. The system of claim 1 wherein the range of angles at which ultrasound waves strike the surface of the biological tissue includes angles from about 60 degrees to about 80 degrees from the axis perpendicular to the surface of the target tissue.

3. The system of claim 1 wherein the ultrasound coupling includes an ultrasound coupling material having lower sound velocities relative to sound velocities of one or more layers of the biological tissue.

4. The system of claim 3 wherein the ultrasound coupling material includes at least one of: water, gel, blood, urine, and other biological fluid.

5. The system of claim 3 wherein the ultrasound coupling material includes a coupling medium whose volume conforms to a change in angles at which ultrasound waves strike the surface of the biological tissue.

6. The system of claim 1 wherein the ultrasound transducer is configured to produce ultrasound waves having at least one of: durations between about 0.001 sec and about 10 minutes; intensities between about 0.001 Watt and about 1000 Watt; and frequencies between about 20 kHz and about 50 MHz.

7. The system of claim 1 wherein the irradiating surface defines a shape to emit ultrasound waves at angles within the range of angles.

8. The system of claim 7 wherein the irradiating surface defines a concave shape.

9. The system of claim 1 wherein the irradiating surface is disposed at an angle.

10. The system of claim 1 wherein the ultrasound transducer is configured to operate in at least one of a pulse mode and a continuous mode.

11. The system of claim 10 including a signal generator operatively coupled to an amplifier, the signal generator and the amplifier being operatively coupled with the ultrasound transducer such that the signal generator and amplifier adjust and control frequencies and intensities of ultrasound waves the ultrasound transducer emits.

12. The system of claim 1 wherein the mechanical force-creating element includes a motor, an electromagnet, or an ultrasonic probe.

13. The system of claim 1 including a central control unit configured and designed to control one or more processes of the elements of the system.

14. The system of claim 13 wherein the central control unit is configured and designed to determine one or more angles within the range of angles at which ultrasound waves strike the surface of the biological tissue based on at least one of: (a) one or more mechanical characteristics of one or more layers of the biological tissue; (b) one or more mechanical characteristics of the fluid; (c) sound velocities of the fluid; and (d) sound velocities of the one or more layers of the biological tissue.

15. The system of claim 1 wherein the fluid container includes a control unit configured to regulate fluid temperatures and to regulate flow rates at which a given volume of the fluid is circulated to and from the housing container.

16. The system of claim 15 wherein the control unit includes a temperature regulator and a pump.

17. The system of claim 1 wherein the sleeve includes a flexible sleeve.

18. The system of claim 17 wherein the flexible sleeve defines a concertina shape.

19. A system for altering activity of at least a portion of one or more layers of biological tissue through application of ultrasound waves, the system comprising:
an ultrasound transducer having an irradiating surface and being constructed and designed to emit ultrasound waves;
a housing container including an interior, the housing contained by configured to dispose within its interior at least a portion of the ultrasound transducer including the irradiating surface and to permit ultrasound waves to emit from the irradiating surface and through a first end of the housing container toward the biological tissue;
a fluid container joined with the housing container such that the interior of the fluid container is in fluid communication with the interior of the housing container, the fluid container being constructed and designed to hold and to circulate a fluid to the interior of the housing container, the fluid at least one of ultrasound coupling and cooling; and
a flexible sleeve on the first end of the housing container through which ultrasound waves emit, the sleeve being configured as singularly adjustable relative to a surface of the biological tissue such that ultrasound waves strike the surface of the biological tissue at angles at least within a range between an irradiation axis of emitted ultrasound waves and an axis perpendicular to the surface of the target tissue so that at least a portion of ultrasound waves convert to and impinge the biological tissue as surface waves that propagate substantially parallel to the surface of the biological tissue.
